(19) United States
(12) Patent Application Publication
(10) Pub. No.: US 2015/0246202 A1
(43) Pub. Date: Sep. 3, 2015

(54) TUBING PACKAGE FOR AN ELONGATE MEDICAL DEVICE AND METHOD MAKING AND USING THE SAME

(71) Applicant: Niall Mcnamara, Co. Clare (IE)

(72) Inventor: Niall Mcnamara, Co. Clare (IE)

(73) Assignee: ADVANT MEDICAL LIMITED, Galaway (IE)

(21) Appl. No.: 14/367,630

(22) PCT Filed: Dec. 20, 2012
(86) PCT No.: PCT/EP2012/076506

§ 371 Date: Jun. 20, 2014

Related U.S. Application Data
(60) Provisional application No. 61/581,911, filed on Dec. 30, 2011.

(51) Int. Cl. A61M 25/00

(52) U.S. Cl. CPC A61M 25/002

(57) ABSTRACT

The invention provides for a package device for an elongate medical device. The device includes a conduit or tube arranged in a coiled configuration. At least one over-molded tubing connector is structured and arranged to maintain the coiled configuration. At least portions of adjacent curved sections of the coiled configuration not covered by the at least one over-molded tubing connector contains substantially no gap or spacing. A method making the device includes arranging a conduit or tube into a coiled configuration inside or within a mold and over-molding plural tubing connectors onto the coiled configuration.

### CROSS-REFERENCE TO RELATED APPLICATIONS

The instant application is a PCT International Application based on U.S. provisional application No. 61/581,911, filed Dec. 30, 2011, the disclosure of which is hereby expressly incorporated by reference hereto in its entirety.

### BACKGROUND OF THE INVENTION

1. Field of the Invention

The invention relates to a tubing package which can be used to house an elongate medical device such as a catheter and/or a guide wire and/or a thrombectomy device, and/or which utilizes one or more features shown in the drawings and/or recited in the claims. A method of making and using the tubing package device is also disclosed.

2. Discussion of Background Information

Tubing packages for storing or packaging an elongate medical device are known. Such devices typically include tubing or lumen which is formed into a circular coiled configuration. The coiled configuration arranges or positions the tubing along a single plane via clips or tubing connectors.

Such packages are typically used to package a guide wire or catheter in a coiled configuration thereby resulting in a compact packaging system and can also safely protect the guide wire or catheter. The packaging can protect the guide wire or catheter in numerous environments such as during manufacture, assembly, packaging, shipping and use.

Such packaging devices include those disclosed in U.S. Pat. No. 7,334,678 to KESLER et al. and US 2011/0,127,186 to ENNS et al. The disclosure of each of these documents is hereby incorporated by reference in its entirety as though set forth in full herein. A tubing packaging device is also known from EP 1 551 715 and/or WO 2004/022433. The disclosure of each of these documents is also hereby incorporated by reference in its entirety as though set forth in full herein.

An improved device, however, would, among other things, make the tubing package more compact and improve upon the devices disclosed in KESLER, ENNS and EP '715. In the case of KESLER, an improvement would reduce or remove the spacing between the tubing coils, more securely attach the tubing connectors by over-molding the tubing connectors onto the coiled tubing, and have tubing connectors which surround the tubing coil package. In KESLER, the clips are attached from an upper or lower side and necessarily produce gaps or spacing between the coils and thus require more clips to retain a given multi-coiled configuration. Because of the spacing, the coils can move relative to each other in the bent plane and be out-of-round in areas between the tubing clips. In the case of ENNS, an improvement would reduce or remove the spacing between the tubing coils, even though ENNS teaches to attach the tubing connectors by over-molding the tubing connectors onto the coiled tubing. In ENNS, the tubing connectors surround each tubing coil, which necessarily produce gaps or spacing between the coils. Because of the spacing, the coils can move relative to each other in the bent plane and be out-of-round in areas between the tubing connectors.

It is submitted that there is a continuing need for a tubing package of the type described herein which has one or more of the above-noted advantages and which are lacking in known devices.

### SUMMARY OF THE INVENTION

According to one non-limiting aspect of the invention, there is provided a package device for an elongate medical device, wherein the device comprises a conduit or tube arranged in a coiled configuration. At least one over-molded tubing connector is structured and arranged to maintain the coiled configuration. At least a portion of adjacent curved sections of the coiled configuration not covered by the at least one over-molded tubing connector contains substantially no gap or spacing. Alternatively or additionally, at least a portion of adjacent curved sections of the coiled configuration not covered by the at least one over-molded tubing connector substantially contact or abut one another.

In embodiments, the elongate medical device is at least one of an elongated wire, an elongated metal rod, a solid stainless steel wire, a medical lumen, a wire insertable within a medical lumen, a catheter and a thrombectomy device.

In embodiments, the over-molded tubing connector retains one of; at least two or three adjacent sections of tubing adjacent one another.

In embodiments, the over-molded tubing connector retains at least one of; three, four or five adjacent sections of tubing adjacent one another.

In embodiments, the over-molded tubing connector retains more than two adjacent sections of tubing adjacent one another and without any gap or spacing therebetween.

In embodiments, the over-molded tubing connector retains four adjacent sections of tubing adjacent one another and without any gap or spacing therebetween.

In embodiments, the over-molded tubing connector comprises plural over-molded tubing connectors and one of said plural over-molded tubing connectors retains three adjacent sections of tubing adjacent one another and another of the plural over-molded tubing connectors retains four adjacent sections of tubing adjacent one another.

In embodiments, the at least one over-molded tubing connector comprises three over-molded tubing connectors each connecting three adjacent sections of tubing adjacent one another along a common plane and one over-molded tubing connector connecting four adjacent sections of tubing adjacent one another along the common plane.

In embodiments, an overall length of the conduit or tube is between about 7 inches and about 100 inches and optionally approximately 60 inches when straight and prior to being arranged in the coiled configuration.

In embodiments, an outer diameter of the coiled configuration is at least one of between about 3 and about 12 inches, between about 5 and about 10 inches, and between about 5 and about 8 inches.

In embodiments, coils of the coiled configuration are arranged one of on a common radial plane and each coil is spaced from a common center axis of the coiled configuration by different amounts.

In embodiments, coils of the coiled configuration are arranged one of axially one in front of another and each coil is spaced from a common center axis of the coiled configuration by substantially a same amount.

According to one non-limiting aspect of the invention, there is provided a package device for an elongate medical device, comprising a conduit or tube arranged in a coiled configuration and plural over-molded tubing connectors being structured and arranged to maintain the coiled configuration. Adjacent curved sections of the coiled configuration between the plural over-molded tubing connectors substantially contact or abut one another.

In embodiments, the elongate medical device is at least one of an elongated wire, an elongated metal rod, a solid stainless steel wire, a medical lumen, a wire insertable within a medical lumen and a catheter.

In embodiments, at least one of the over-molded tubing connectors retains at least three adjacent sections of tubing adjacent one another and without any gap or spacing therebetween.

In embodiments, an overall length of the conduit or tube is between about 7 inches and about 100 inches and optionally approximately 60 inches when straight and prior to being arranged in the coiled configuration.

In embodiments, an outer diameter of the coiled configuration is at least one of between about 3 and about 12 inches, between about 5 and about 10 inches, and between about 5 and about 8 inches.

According to one non-limiting aspect of the invention, there is provided a method making the device of anyone of the types described above, wherein the method comprises arranging a conduit or tube into a coiled configuration inside or within a mold and over-molding plural tubing connectors onto the coiled configuration.

According to one non-limiting aspect of the invention, there is provided a method making a package device for an elongate medical device, wherein the method comprises arranging a conduit or tube in a coiled configuration and over-molding at least one tubing connector onto the coiled configuration. After the over-molding, adjacent curved sections of the coiled configuration substantially contact or abut one another.

In embodiments, the over-molding comprises over-molding plural spaced apart tubing connectors onto the coiled configuration and adjacent curved sections of the coiled configuration arranged between the plural over-molded tubing connectors substantially contact or abut one another.

According to one non-limiting aspect of the invention, there is provided a tubing package that is more compact and improves upon the devices known in the art. In particular, the device reduces or removes the spacing between the tubing coils, more securely attaches the tubing connectors by over-molding the tubing connectors onto the coiled tubing, and utilizes tubing connectors which surround the tubing coil package while allowing adjacent cured sections of the tube to substantially abut or contact one another. Each bent section thus serves to reinforce an adjacent bent section and vice versa. Moreover, the tubing connectors are non-removable and less of them are required—unlike known packages which utilize attachable clips. Moreover, by removing the spacing, the coils generally cannot move relative to each other in the bent or common plane. As such, the problem of out-of-round areas between the tubing connectors is reduced or eliminated.

According to another non-limiting aspect of the invention, there is provided a package device for an elongate medical device, comprising a conduit or tube arranged in a coiled configuration and at least two over-molded tubing connectors being structured and arranged to maintain the coiled configuration. At least portions of adjacent curved sections of the coiled configuration between the at least two over-molded tubing connectors contain substantially no gap or spacing.

In embodiments, the elongate medical device is at least one of; an elongated wire, an elongated metal rod, a solid stainless steel wire, a medical lumen, a wire insertable within a medical lumen, and a catheter.

In embodiments, at least one of the over-molded tubing connectors retains three adjacent sections of tubing adjacent one another.

In embodiments, at least one of the over-molded tubing connectors retains four adjacent sections of tubing adjacent one another.

In embodiments, at least one of the over-molded tubing connectors retains three adjacent sections of tubing adjacent one another and without any gap or spacing therebetween.

In embodiments, at least one of the over-molded tubing connectors retains four adjacent sections of tubing adjacent one another and without any gap or spacing therebetween.

In embodiments, one of the over-molded tubing connectors retains three adjacent sections of tubing adjacent one another and another of the over-molded tubing connectors retains four adjacent sections of tubing adjacent one another.

In embodiments, the at least two over-molded tubing connectors comprises; three over-molded tubing connectors each connecting three adjacent sections of tubing adjacent one another along a common plane, and one over-molded tubing connector retains four adjacent sections of tubing adjacent one another along the common plane.

In embodiments, an overall length of the conduit or tube is between about 7 inches and about 100 inches and optionally approximately 60 inches when straight and prior to being arranged in the coiled configuration.

In embodiments, an outer diameter of the coiled configuration is at least one of; between about 3 and about 12 inches, between about 5 and about 10 inches, and between about 5 and about 8 inches.

According to another non-limiting aspect of the invention, there is provided a package device for an elongate medical device, comprising a conduit or tube arranged in a coiled configuration along a common plane and plural over-molded tubing connectors being structured and arranged to maintain the coiled configuration. Adjacent curved sections of the coiled configuration between the plural over-molded tubing connectors substantially contact or abut one another.

In embodiments, the elongate medical device is at least one of; an elongated wire, an elongated metal rod, a solid stainless steel wire, a medical lumen, a wire insertable within a medical lumen, and a catheter.

In embodiments, at least one of the over-molded tubing connectors retains three adjacent sections of tubing adjacent one another.

In embodiments, at least one of the over-molded tubing connectors retains four adjacent sections of tubing adjacent one another.

In embodiments, at least one of the over-molded tubing connectors retains three adjacent sections of tubing adjacent one another and without any gap or spacing therebetween.

In embodiments, at least one of the over-molded tubing connectors retains four adjacent sections of tubing adjacent one another and without any gap or spacing therebetween.

In embodiments, one of the over-molded tubing connectors retains three adjacent sections of tubing adjacent one another and another of the over-molded tubing connectors retains four adjacent sections of tubing adjacent one another.

In embodiments, the plural over-molded tubing connectors comprise three over-molded tubing connectors each connecting three adjacent sections of tubing adjacent one another along a common plane and one over-molded tubing connector retains four adjacent sections of tubing adjacent one another along the common plane.

In embodiments, an overall length of the conduit or tube is between about 7 inches and about 100 inches and optionally approximately 60 inches when straight and prior to being arranged in the coiled configuration.

In embodiments, an outer diameter of the coiled configuration is at least one of; between about 3 and about 12 inches, between about 5 and about 10 inches, and between about 5 and about 8 inches.

According to another non-limiting aspect of the invention, there is provided a package device for an elongate medical device, comprising a conduit or tube arranged in a coiled configuration along a common plane and plural over-molded tubing connectors being structured and arranged to maintain the coiled configuration. The coiled configuration comprises an inner coil, at least one intermediate coil and an outer coil. Each of the inner, intermediate and outer coils are arranged on the common plane. Adjacent curved sections of the coiled configuration between the plural over-molded tubing connectors substantially contact or abut one another.

In embodiments, adjacent curved sections of the coiled configuration connected by the plural over-molded tubing connectors substantially contact or abut one another.

According to another non-limiting aspect of the invention, there is provided a method of making the device of any of the types described above, wherein the method comprises arranging the conduit or tube into a coiled configuration within a mold and over-molding plural tubing connectors onto the coiled configuration.

According to another non-limiting aspect of the invention, there is provided a method of using the device of any of the types described above, wherein the method comprises inserting one end of an elongate medical device into one end of the coiled configuration.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a top view of a first non-limiting embodiment of a tubing package;

FIG. 2 shows a front side cross-section view of FIG. 1 along Section A-A. The tubes within the tubing connectors are not shown to illustrate the little to no wall thickness between the adjacent tube sections within the connectors;

FIG. 3 shows an enlarged part of FIG. 2 labeled Detail B;

FIG. 4 shows a left side view of FIG. 1 or a view 90 degrees from that of FIG. 2;

FIG. 5 shows the tube or conduit used in FIG. 1 in a straight orientation and in partial cross-section;

FIG. 6 shows an end view of the tube or conduit of FIG. 5;

FIG. 7 shows a top view of a second non-limiting embodiment of a tubing package;

FIG. 8 shows a top perspective view of a third non-limiting embodiment of a tubing package and illustrates tubing that is substantially transparent or translucent;

FIG. 9 shows a top view of a fourth non-limiting embodiment of a tubing package;

FIG. 10 shows a partial enlarged view of FIG. 9;

FIG. 11 shows a top perspective view of a still another non-limiting embodiment of a tubing package and illustrates tubing whose coils are oriented axially instead of on a common radial plane;

FIG. 12 shows a partial enlarged view of FIG. 11;

FIG. 13 shows a side view of FIG. 11;

FIG. 14 shows an empty mold half that can be used to over-mold connectors onto a coil of tubing in order to form a tubing package in accordance with one non-limiting embodiment of the invention;

FIG. 15 shows the mold half of FIG. 14 with one end of tubing being inserted therein;

FIGS. 16 and 17 show the mold half of FIG. 14 as the tubing is almost fully inserted therein and fully inserted therein;

FIGS. 18 and 19 shows the mold half of FIG. 17 with the tubing coiled therein and both before and after a second mold half is moved into contact or engagement with the first mold half;

FIG. 20 shows a cross-section view of the closed mold of FIG. 19 and illustrates how plastic resin material can be injected into the mold so as to form the over-molded connectors;

FIG. 21 shows the mold of FIG. 19 after it is opened and after the over-molded connectors have been formed and cooled sufficiently;

FIG. 22 shows the tubing package being removed from the opened mold of FIG. 20;

FIGS. 23 and 24 show views of an exemplary non-limiting tubing package formed in the mold system of FIGS. 14-20; and

FIG. 25 illustrates an exemplary process of forming the tubing package using the mold system of FIGS. 14-20.

### DETAILED DESCRIPTION OF THE INVENTION

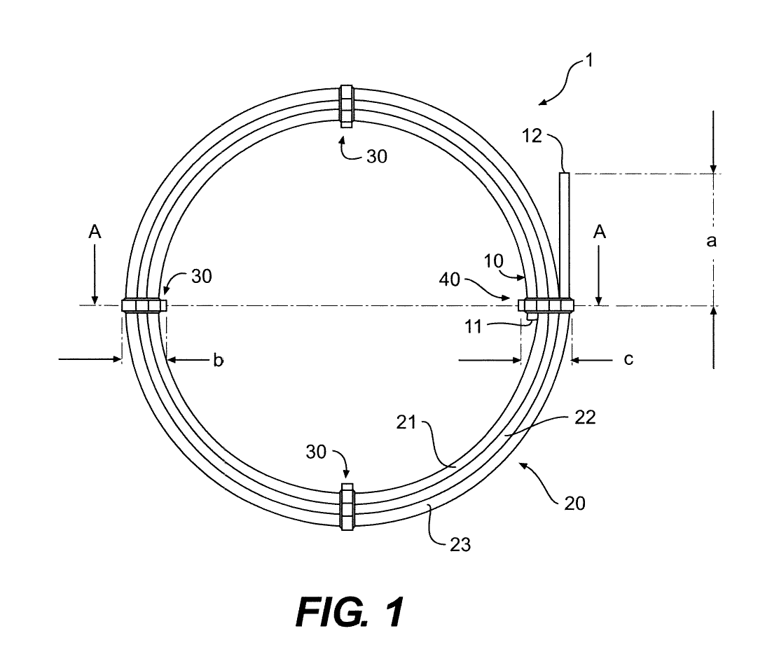

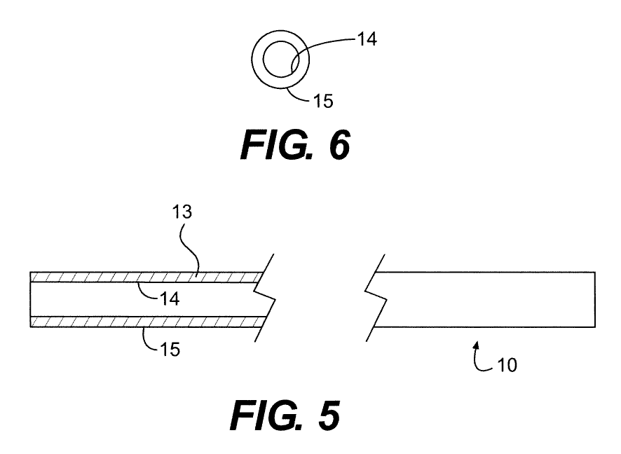

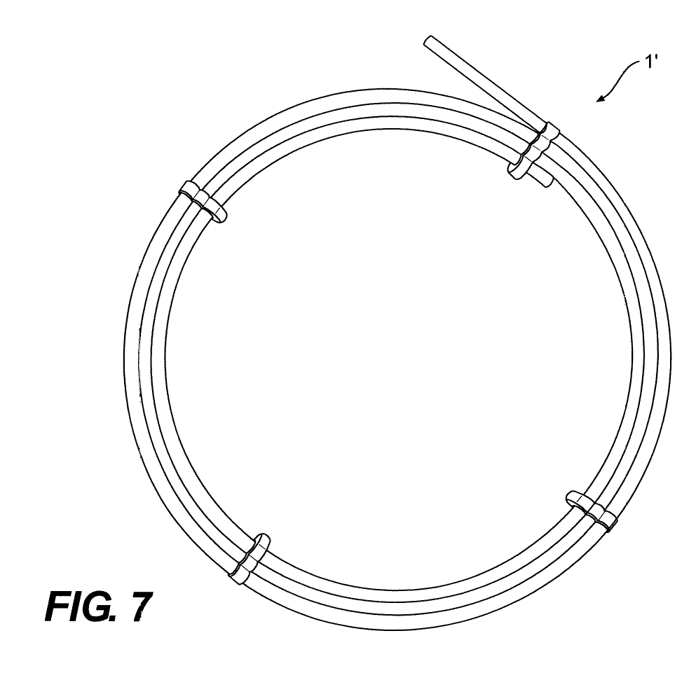

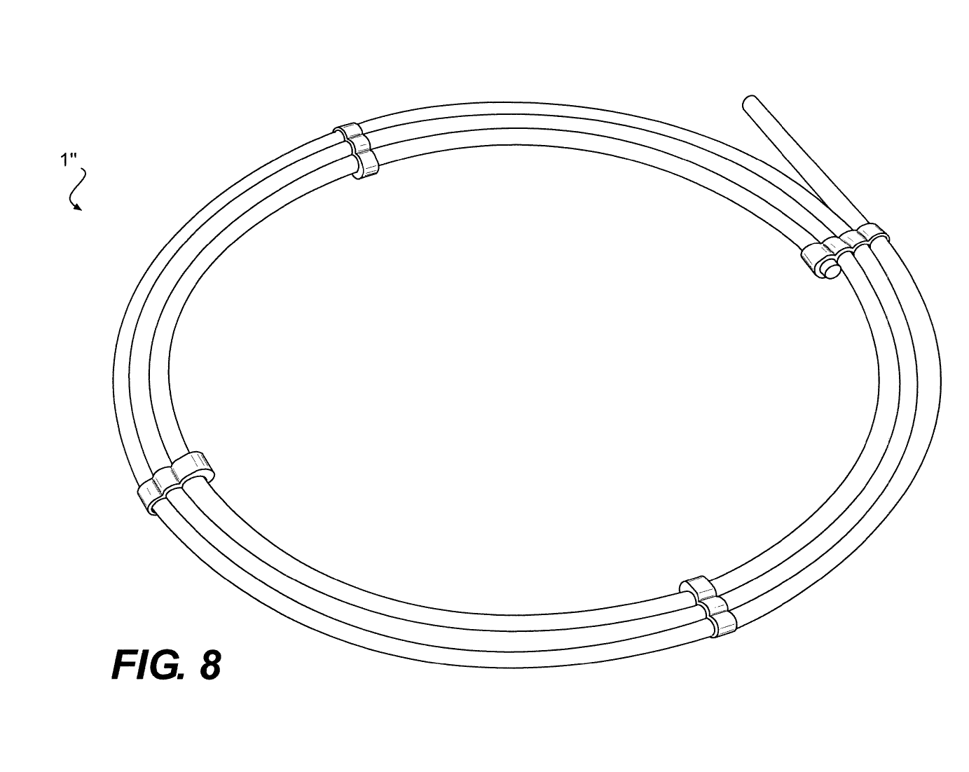

FIGS. 1-7 show a first non-limiting embodiment of a tubing package or package device 1. The package 1 is formed as a coil 20 of tubing 10 which, when straight and prior tom being coiled, can have an overall length. In one non-limiting embodiment, the overall length is between about 7 inches and about 100 inches and optionally about 60 inches. The tubing 10 has a first end 11 which can be an open end. Alternatively, end 11 can be closed or partially closed. The tubing 10 also has a second end 12 which is an open end and which can receive therein and allow for insertion therein of an item that it to be packaged within the coil 20. As is apparent from FIGS. 6 and 7, the tubing 10 has a wall 13 which serves to protect and seal with the item or package disposed within. The wall 13 has inside generally cylindrical surface 14 as well as an outside generally cylindrical surface 15. The inside surface 14 defines within it a space which should be sized and configured to receive therein the item and should allow for easy, unobstructed and full or complete insertion therein of the item.

As can be seen in FIG. 1, the coil of tubing 20 can include an inner winding 21 having the form of a generally curved section, e.g., circular, of tubing. The inner winding 21 is defined by the first end 11 and transitions into an intermediate winding 22 in an area where it is disposed adjacent the first end 11. The intermediate winding 22 is defined by the previous transition area and transitions into an outer winding 23 in an area where it is disposed adjacent the first end 11. The outer winding 23 is defined by the previous transition area and extends to the second end 12. In embodiments, a portion of the tubing 10 extending between the second end 12 and where the tubing meets the coil section 20 is made to have a generally straight configuration for a desired length “a” as shown in FIG. 1. This straight section can serve to, among other things, facilitate insertion of the item or device being packaged.

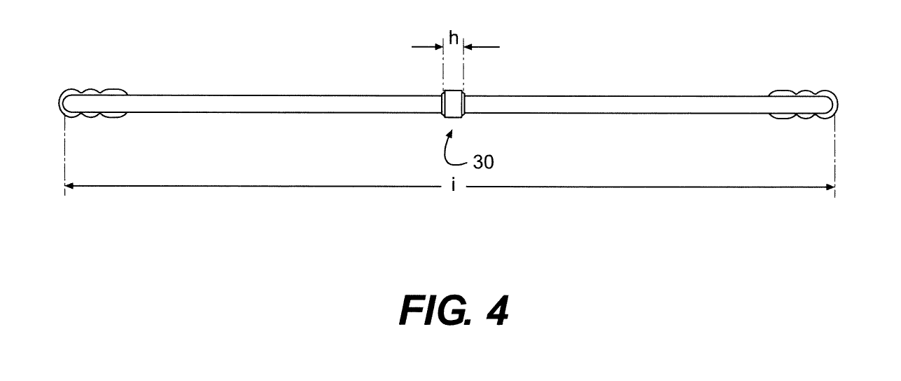

Again with reference to FIG. 1, the coil of tubing 20 can be maintained in this coiled configuration via one or more over-molded connectors 30. In the non-limiting embodiment shown in FIG. 1, three over-molded connectors 30 are each angularly spaced by, e.g., approximately 90 degrees. An additional over-molded connector 40 is angularly spaced from one or more of the connectors 30 by, e.g., approximately 90 degrees. Also, in the non-limiting embodiment shown in FIG. 1, the additional over-molded connector 40 serves as an area for locating the tubing ends 11 and 12. In FIG. 1, the three over-molded connectors 30 couple together three coil sections 21-23 whereas the additional over-molded connector 40 couples together the coil sections in an area of the opposite ends 11 and 12. As can be seen in FIG. 4, an outermost diameter of the coil package 1 (measured to an outermost part of the connectors) can have value “i”. Moreover, a width of each connector 30/40 can have value

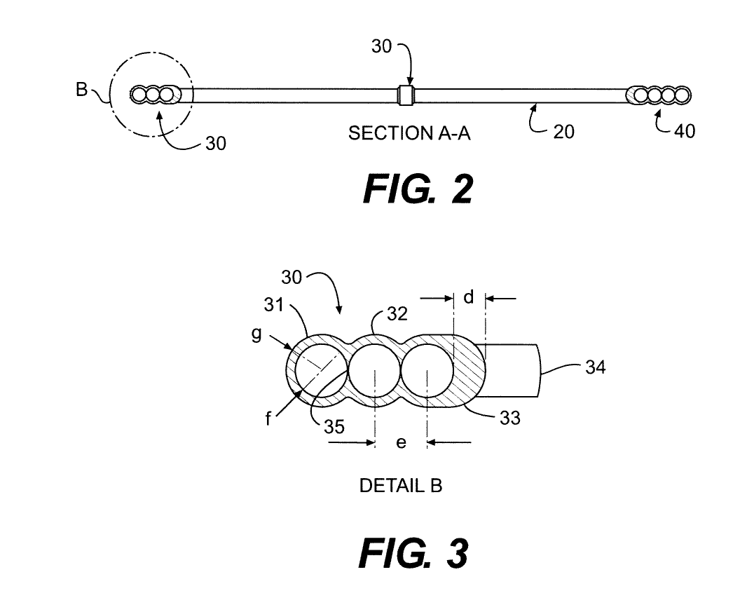

As is apparent in FIG. 1 and as is made more so in FIGS. 2 and 3, the coil of tubing 20 arranges or couples together three coil sections 21-23 on a common plane such that a diameter of each coil section increases between sections 21-23. Furthermore, the connector 30 connects together the coil sections such that substantially abut or contact one another as shown in FIG. 3. Moreover, as shown in FIG. 3, section 31 of the connector 30 surrounds most of the tubing outer diameter of part of section 21, but does not extend into substantially the tubing outer surface contact area 35 between section 21 and 22. Section 32 of the connector 30 surrounds only opposite sides of the tubing outer diameter of part of section 22, but does not extend into substantially the tubing outer surface contact areas 35 between sections 21 and 22 and between sections 22 and 23. Section 33 of the connector 30 surrounds most of the tubing outer diameter of part of section 23, but does not extend into substantially the tubing outer surface contact area 35 between section 22 and 23. Because of the sections 21-23 contact each other at contact area 35, the space between these sections is essentially zero (or close thereto so that only a thin amount of connector material can extend into the area 35) within the connectors 30 and 40, and also in areas of the coil 20 which lack connectors 30/40, i.e., the curved sections of the coil 20 between the connectors 30/40. As a result of the configuration shown in FIGS. 1-3, connector 30 has a width “b” and connector 40 has a width “c”. Connectors 30 and 40 also have an inner surface defined by radius “f”, an outer surface defined by radius “g”, a width defined by dimension “d” and a center to center spacing defined by dimension “e”.

According to one non-limiting example, the packaging device 1 can have the following values. Dimension “a” is, e.g., about 2 inches. Dimension “b” is, e.g., about 0.6 inches. Dimension “c” is, e.g., about 0.76 inches. Dimension “d” is, e.g., about 0.1 inches. Dimension “e” is, e.g., about 0.156 inches. Dimension “f” is, e.g., about 0.156 inches in diameter. Dimension “g” is, e.g., about 0.11 inches in radius. Dimension “h” is, e.g., about 0.157 inches. Dimension “i” is, e.g., about 6.6 inches. The tube 10 may be extruded in a uniform or tapered cross section to accommodate various surgical device configurations, and may have an overall length that typically exceeds that of the surgical device. Thus, the tube 10 may have a length in a range slightly greater than about 7 and 200 inches and also between about 7 inches and about 100 inches. The lumen may have a diameter 14 of about 0.030 inches to about 0.300 inches. An outer diameter 15 of the tube 10 can typically range between about 0.100 inches and about 0.500 inches. A typical wall thickness of the tube 10 can be in a range between about 0.010 inches and 0.100 inches.

FIG. 7 shows a second non-limiting embodiment of a tubing package 1′. This embodiment is similar to that of FIG. 1, but shows how the tubing coil and connectors can be made to have different colors and/or shade of color and/or material. In embodiments, the material of the tubing is such that any item inserted therein need not be visible through the wall of the tubing. In embodiments, the material of the tubing is such that any item inserted therein can be visible through the wall of the tubing by making the wall substantially transparent or translucent.

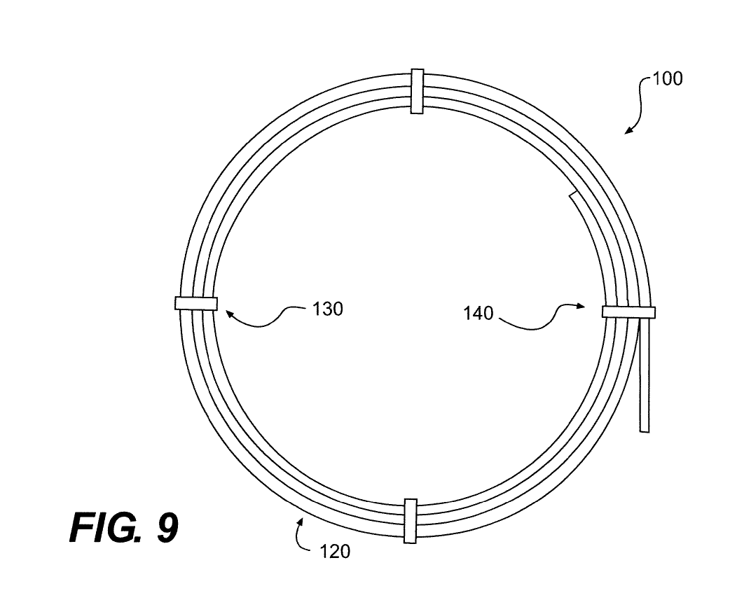

FIG. 8 shows such a non-limiting embodiment of a tubing package 1″ and illustrates tubing that is substantially transparent or translucent.

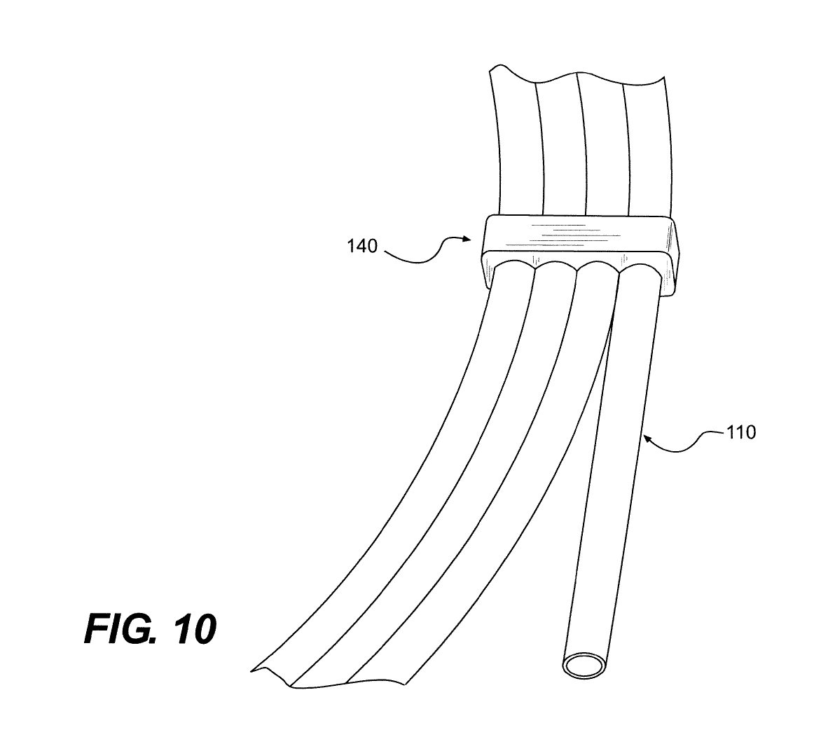

FIGS. 9 and 10 show another non-limiting embodiment of a tubing package 100. This embodiment is similar to that shown in FIG. 1 except that the first or inside end of the tubing 110 extends past the connector 140 by a greater amount and utilizes different configured connectors 130 and 140. As can be seen in FIG. 10, the connector has a generally rectangular outer configuration. Otherwise, the package 100 utilizes a tubing coil 120 having plural substantially equally spaced over-molded connectors 130 and 140.

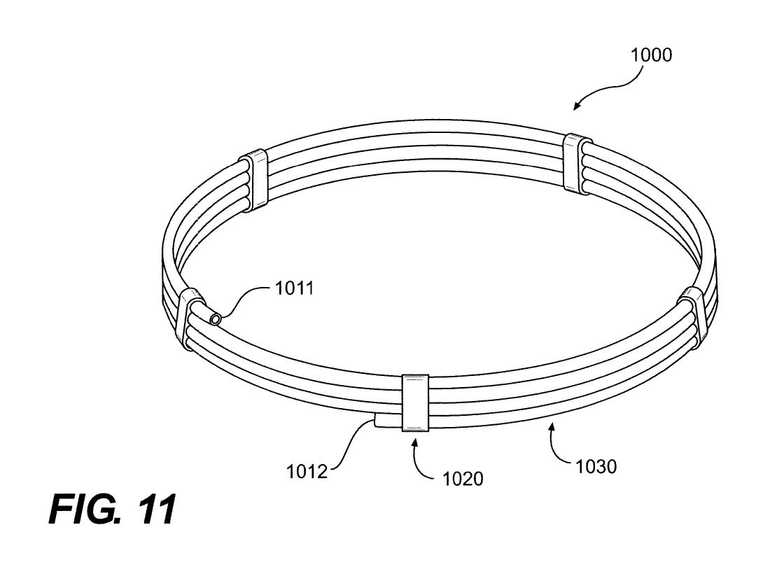

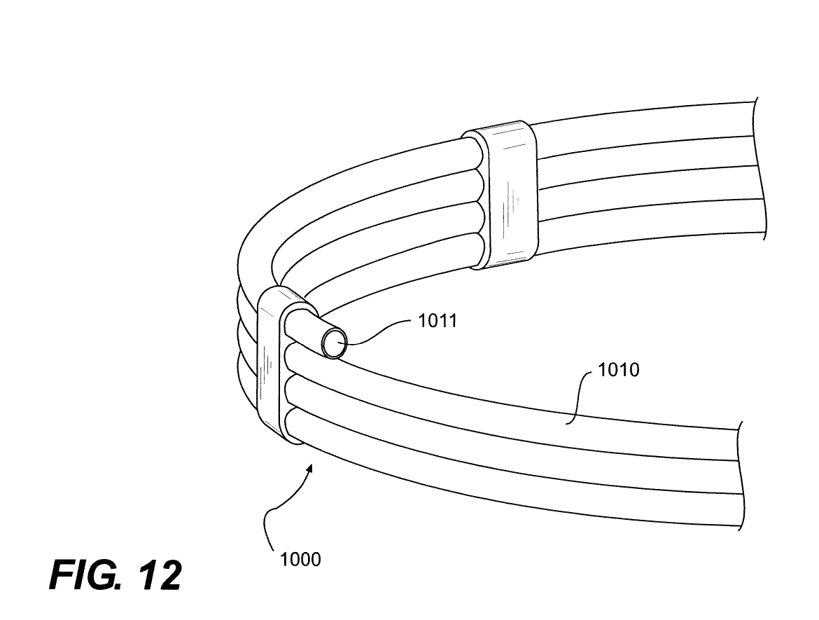

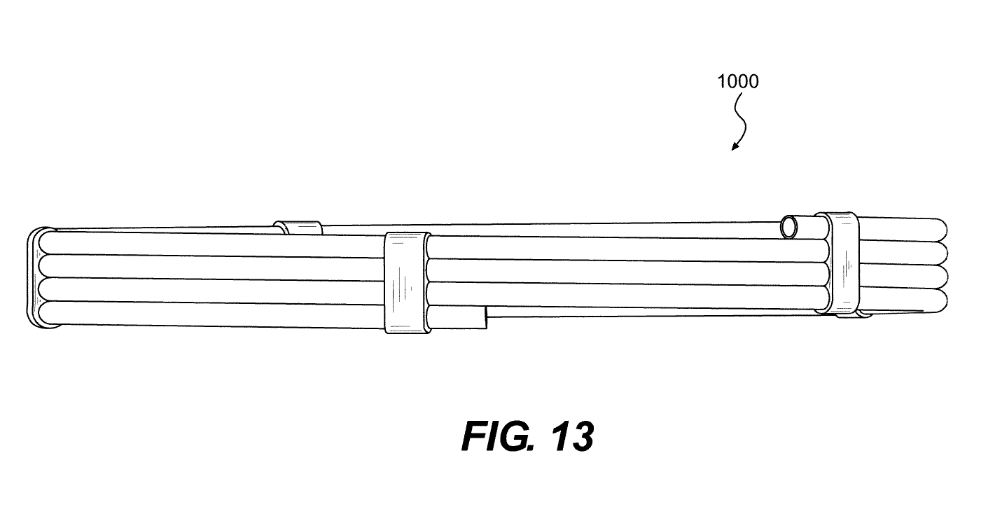

FIGS. 11-13 show still another non-limiting embodiment of a tubing package or package device 1000. The package 1000 is formed as a coil 1020 of tubing 1010 which, when straight and prior tom being coiled, can have an overall length. In one non-limiting embodiment, the overall length is between about 7 inches and about 100 inches and optionally about 60 inches. The tubing 1010 has a first end 1011 which can be an open end. Alternatively, end 1011 can be closed or partially closed. The tubing 1010 also has a second end 1012 which is an open end and which can receive therein and allow for insertion therein of an item that it to be packaged within the coil 1020. As is previous embodiments, the tubing 1010 has a wall which serves to protect and seal with the item or package disposed within. The wall has inside generally cylindrical surface as well as an outside generally cylindrical surface. The inside surface defines within it a space which should be sized and configured to receive therein the item and should allow for easy, unobstructed and full or complete insertion therein of the item.

In this embodiment, rather than the coils of the coiled configuration being arranged on a common radial plane or with each coil being spaced from a common center axis of the coiled configuration by different amounts, the coil sections of the coil of tubing 1020 are arranged axially one in front of another and/or each coil section is spaced from a common center axis of the coiled configuration by substantially a same amount as shown in FIGS. 11-13. Furthermore, the first end 1011 is arranged in an area of one of the connectors 1030 while the second end 1012 is arranged in an area of another connector 1030 and both ends 1011 and 1012 face or are oriented toward one another. Furthermore, each connector 1030 couples together four or equal numbers of coil sections, has two oppositely arranged generally planar sides, and two oppositely arranged outwardly curved sides.

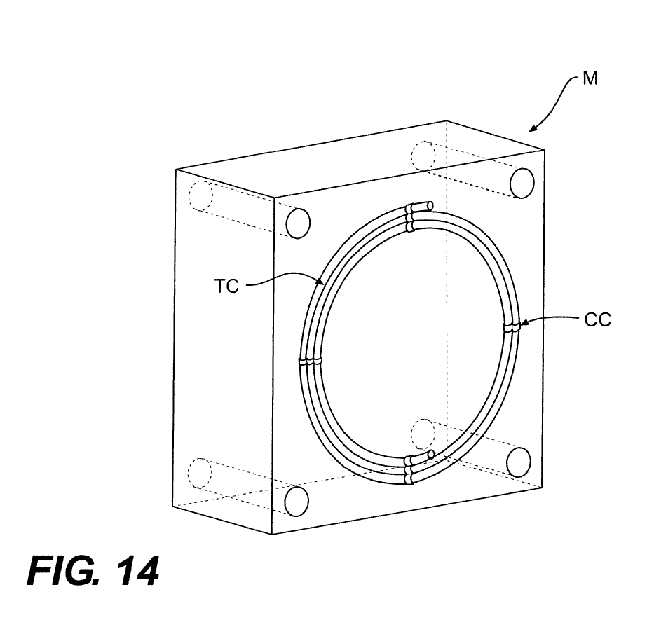

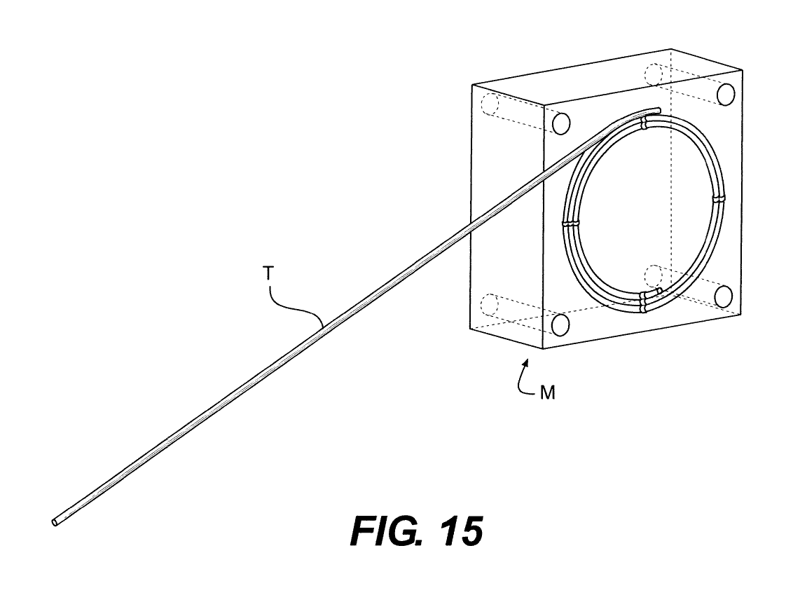

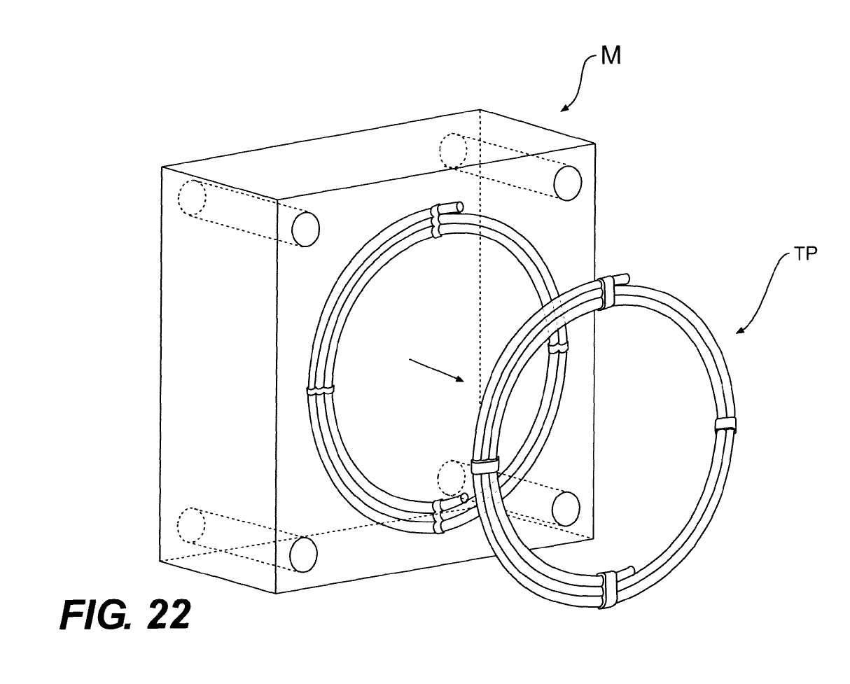

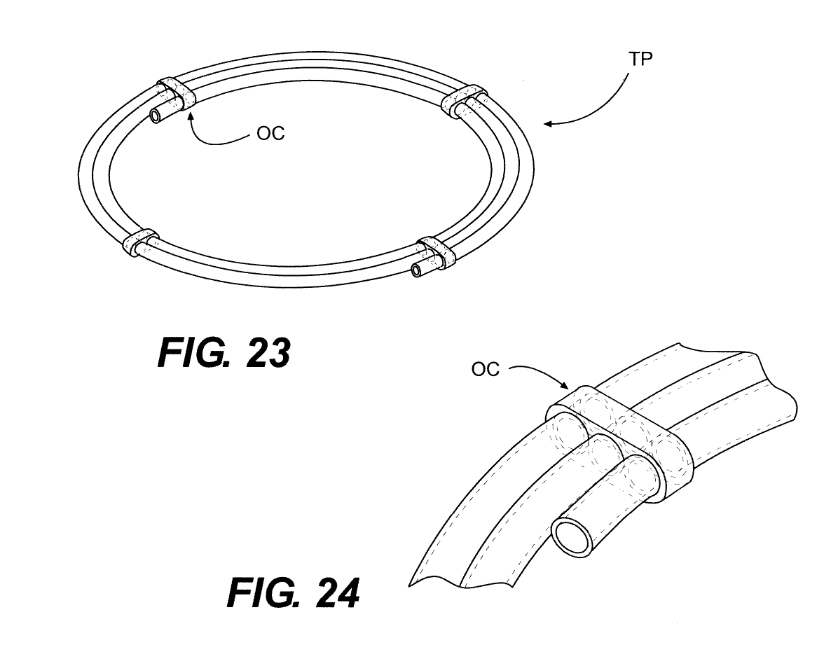

FIGS. 14-22 and 25 show one non-limiting way in which the tubing package of FIGS. 23 and 24 can be manufactured and/or how the coil of tubing can be over-molded with plural connectors using a mold system. This mold system and process can also be utilized to make any of the herein disclosed embodiments such as those shown in FIGS. 1-13.

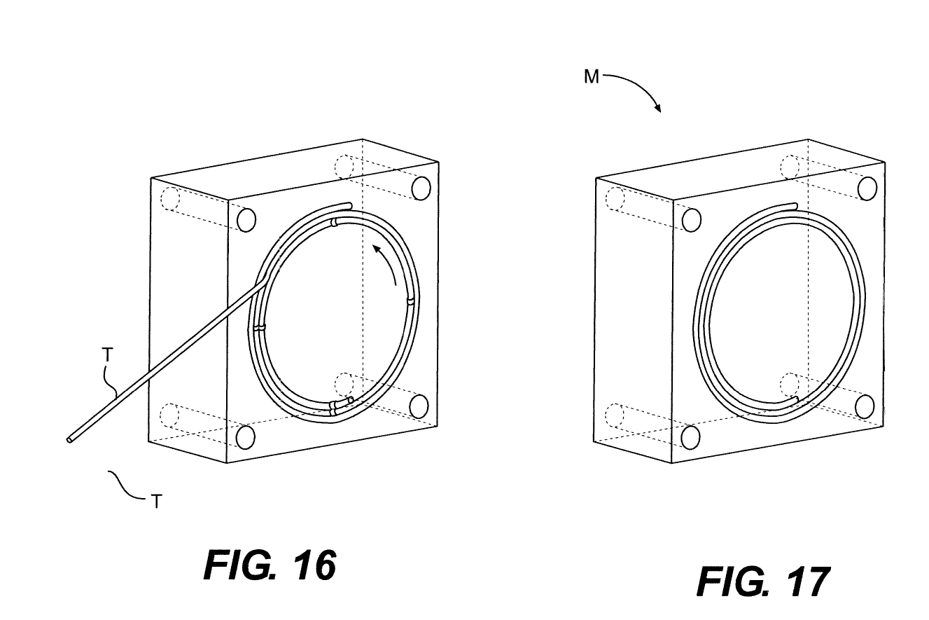

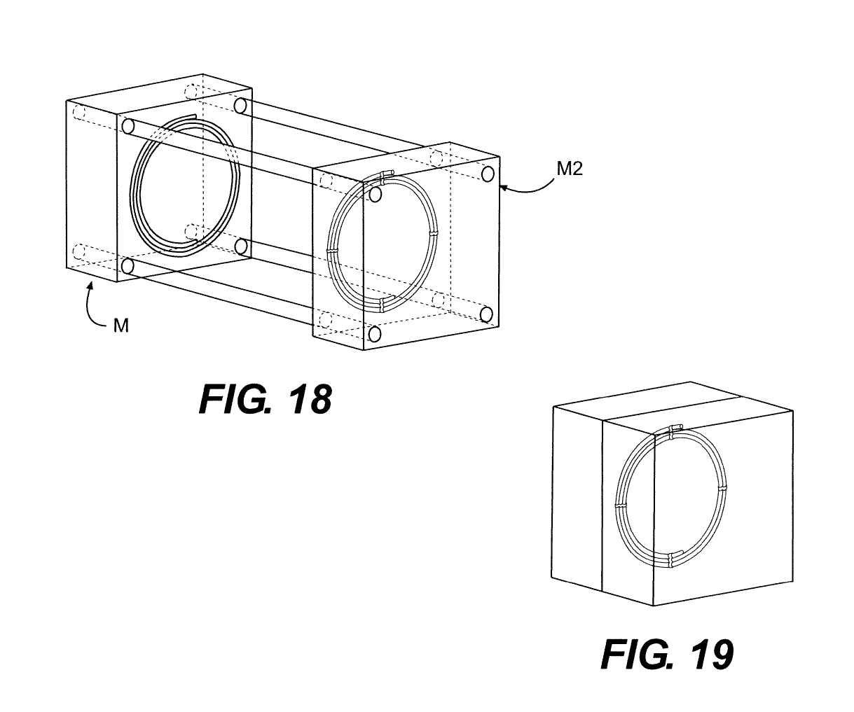

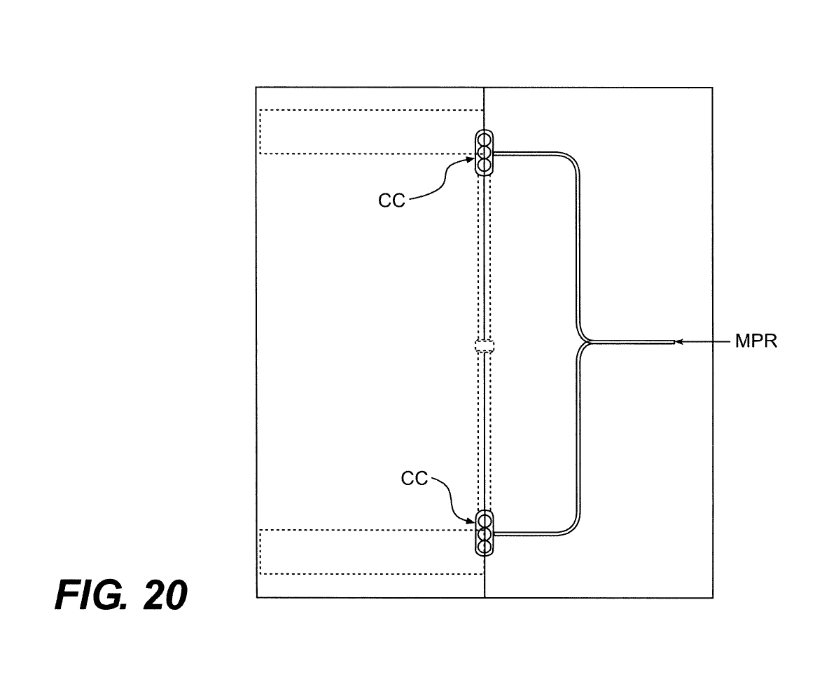

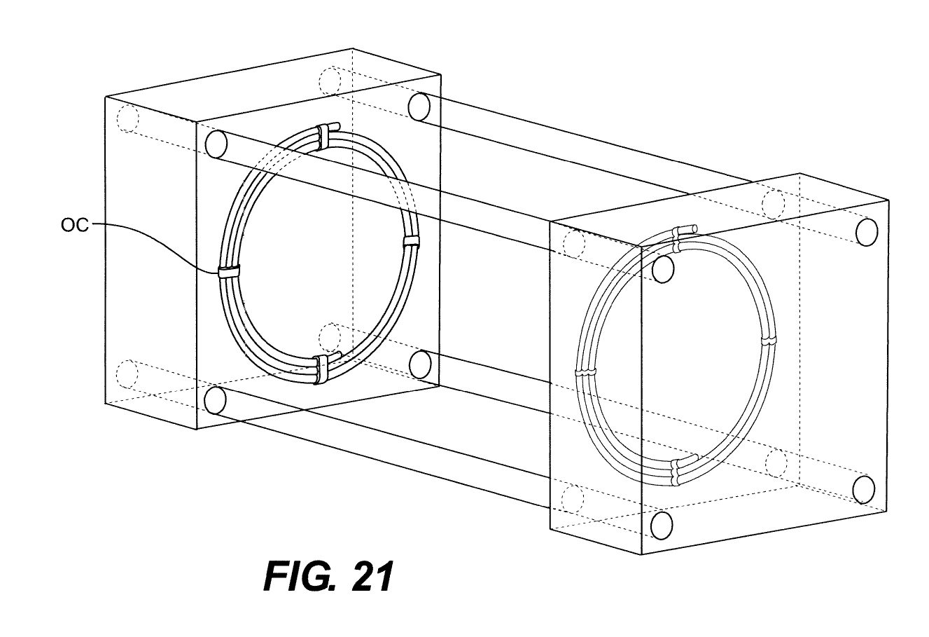

FIG. 14 shows an empty mold half M that can be used to over-mold connectors onto a coil of tubing in order to form a tubing package in accordance with one non-limiting embodiment of the invention. The mold M includes a spiral shaped tubing cavity TC sized and configured to receive therein the coil of tubing. Also formed in the mold M are over-molded connector cavities CC located in specific desired locations. With reference to FIG. 16, it can be seen how a specific length of tubing T can be initially positioned within the mold M and coiled into the cavity TC (or the mold rotated to coil the tubing). FIGS. 16 and 17 show the mold M as the tubing T is almost fully inserted therein and fully inserted therein. Next, in FIGS. 18 and 19, it can be seen how a second mold half M2, spaced from the mold M with the tubing coiled therein, can be moved into contact or engagement with the mold half M. As can be seen in FIG. 20, once both mold half are brought into contact or closed, molten plastic resin material MPR can be injected into the mold and directed to the connector cavities CC so as to form the over-molded connectors OC shown in FIG. 21. It should be noted that the injection location shown in FIG. 20 for injecting the material MPR can be in any desired area or portion of the mold system and can be multiple injection locations, and is not limited to that shown in FIG. 20. FIG. 21 shows the mold of FIGS. 19 and 20 after it is opened and after the over-molded connectors OC have been formed and cooled sufficiently. Next, as shown in FIG. 21, the tubing package TP is removed from the mold M. With the exception of the over-molding stages, the coil of tubing can also be made in a manner similar to that disclosed in WO 2004/022433 already expressly incorporated by reference herein in its entirely.

FIGS. 23 and 24 show views of an exemplary non-limiting tubing package TP formed in the mold system of FIGS. 14-20. This embodiment is similar to the embodiments of FIGS. 1-13, and locates the free opposite ends in areas of different oppositely arranged over-molded connectors OC.

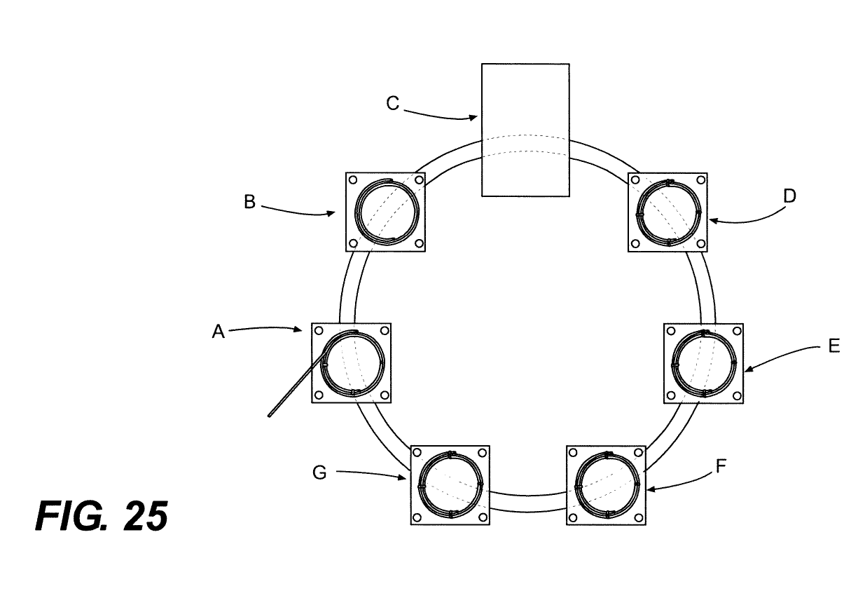

FIG. 25 illustrates an exemplary process of forming the tubing package using the mold system of FIGS. 14-20. In stage A, the tubing is inserted or loaded into the tubing cavity of a mold. Next, in stage B, a checked is performed to determine if the properly loaded tubing is presented in the mold. In stage C, the mold is placed in an over-molding machine and plastic resin material is injected into the mold. In stage D, the mold is allowed to cool and opened. In stage E, a visual quality control inspection is performed. Next, in stage F, the tubing package is ejected from the mold. In final stage G, the empty mold is made ready to be used in stage A.

It is noted that the foregoing examples have been provided merely for the purpose of explanation and are in no way to be construed as limiting of the present invention. While the present invention has been described with reference to one or more exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. A package device for an elongate medical device, comprising:
a conduit or tube arranged in a coiled configuration;
at least one over-molded tubing connector being structured and arranged to maintain the coiled configuration; and
at least one of:
a portion of adjacent curved sections of the coiled configuration not covered by the at least one over-molded tubing connector contains substantially no gap or spacing; and
a portion of adjacent curved sections of the coiled configuration not covered by the at least one over-molded tubing connector substantially contact or abut one another.

2. The device of claim 1, wherein the elongate medical device is at least one of:
an elongated wire;
an elongated metal rod;
a solid stainless steel wire;
a medical lumen;
a wire insertable within a medical lumen; and
a catheter.

3. The device of claim 1, wherein said over-molded tubing connector retains three adjacent sections of tubing adjacent one another.

4. The device of claim 1, wherein said over-molded tubing connector retains four adjacent sections of tubing adjacent one another.

5. The device of claim 1, wherein said over-molded tubing connector retains three adjacent sections of tubing adjacent one another and without any gap or spacing therebetween.

6. The device of claim 1, wherein said over-molded tubing connector retains four adjacent sections of tubing adjacent one another and without any gap or spacing therebetween.

7. The device of claim 1, wherein said over-molded tubing connector comprises plural over-molded tubing connectors and one of said plural over-molded tubing connectors retains three adjacent sections of tubing adjacent one another and another of the plural over-molded tubing connectors retains four adjacent sections of tubing adjacent one another.

8. The device of claim 1, wherein the at least one over-molded tubing connector comprises:
three over-molded tubing connectors each connecting three adjacent sections of tubing adjacent one another along a common plane; and
one over-molded tubing connector connecting four adjacent sections of tubing adjacent one another along the common plane.

9. The device of claim 1, wherein an overall length of the conduit or tube is approximately 60 inches when straight and prior to being arranged in the coiled configuration.

10. The device of claim 1, wherein an outer diameter of the coiled configuration is at least one of:
between about 3 and about 12 inches;
between about 5 and about 10 inches; and
between about 5 and about 8 inches.

11. The device of claim 1, wherein coils of the coiled configuration are arranged one of:
on a common radial plane; and
each coil is spaced from a common center axis of the coiled configuration by different amounts.

12. The device of claim 1, wherein coils of the coiled configuration are arranged one of:
axially one in front of another; and
each coil is spaced from a common center axis of the coiled configuration by substantially a same amount.

13. A package device for an elongate medical device, comprising:
a conduit or tube arranged in a coiled configuration; and
plural over-molded tubing connectors being structured and arranged to maintain the coiled configuration,
wherein adjacent curved sections of the coiled configuration between the plural over-molded tubing connectors substantially contact or abut one another.

14. The device of claim 13, wherein the elongate medical device is at least one of:
an elongated wire;
an elongated metal rod;
a solid stainless steel wire;
a medical lumen;
a wire insertable within a medical lumen; and
a catheter.

15. The device of claim 13, wherein at least one of the over-molded tubing connectors retains at least three adjacent sections of tubing adjacent one another and without any gap or spacing therebetween.

16. The device of claim 13, wherein an overall length of the conduit or tube is approximately 60 inches when straight and prior to being arranged in the coiled configuration.

17. The device of claim 13, wherein an outer diameter of the coiled configuration is at least one of:
between about 3 and about 12 inches;
between about 5 and about 10 inches; and
between about 5 and about 8 inches.

18. A method making the device of claim 1, the method comprising:
arranging a conduit or tube into a coiled configuration inside or within a mold; and
over-molding plural tubing connectors onto the coiled configuration.

19. A method making a package device for an elongate medical device, comprising:
arranging a conduit or tube in a coiled configuration;
over-molding at least one tubing connector onto the coiled configuration, wherein, after the over-molding, adjacent curved sections of the coiled configuration substantially contact or abut one another.

20. The method of claim 19, wherein the over-molding comprises over-molding plural spaced apart tubing connectors onto the coiled configuration and adjacent curved sections of the coiled configuration arranged between the plural over-molded tubing connectors substantially contact or abut one another.
